(19) United States
(12) Patent Application Publication
(10) Pub. No.: US 2023/0201314 A1
(43) Pub. Date: Jun. 29, 2023

(54) USE OF ADVANCED PLATELET-RICH FIBRIN IN PREPARING MEDICAMENT FOR TREATMENT OF DIABETIC FOOT ULCER

(71) Applicant: Air Force Medical Center, PLA, Beijing (CN)

(72) Inventors: Di ZHU, Beijing (CN); Caizhe YANG, Beijing (CN); Nan SU, Beijing (CN); Hongmei CHEN, Beijing (CN); Li XIAO, Beijing (CN); Ying CHEN, Beijing (CN); Liangchen WANG, Beijing (CN); Chenrui WANG, Beijing (CN); Xiaotian ZHANG, Beijing (CN)

(21) Appl. No.: 18/145,278

(22) Filed: Dec. 22, 2022

(30) Foreign Application Priority Data
Dec. 27, 2021 (CN) 202111614946.3

(51) Int. Cl. A61K 38/36; A61P 3/10; A61P 17/02; A61K 35/19

(52) U.S. Cl. CPC A61K 38/36; A61P 3/10; A61P 17/02; A61K 35/19; A61K 2035/124

(57) ABSTRACT

The present disclosure relates to the technical field of biomedicine, in particular to use of advanced platelet-rich fibrin (A-PRF) in preparing a medicament for the treatment of diabetic foot ulcer. The A-PRF provided by the present disclosure has a loose reticular fibrin structure, can be rich in more platelets and leukocytes, can release a plurality of growth factors and cytokines more persistently, and is more beneficial to tissue regeneration and wound repair. The medicament prepared from the A-PRF shortens the healing period of the diabetic foot ulcer, improves the healing rate, and has an excellent treatment effect and high safety.

### CROSS REFERENCE TO RELATED APPLICATION

This patent application claims the benefit and priority of Chinese Patent Application No. 202111614946.3, filed with the China National Intellectual Property Administration on Dec. 27, 2021, the disclosure of which is incorporated by reference herein in its entirety as part of the present application.

### TECHNICAL FIELD

The present disclosure relates to the technical field of biomedicine, in particular to use of advanced platelet-rich fibrin (A-PRF) in preparing a medicament for the treatment of diabetic foot ulcer.

### BACKGROUND

Diabetic foot refers to the destruction of the skin and its deep tissue caused by diabetic neuropathy and/or vasculopathy of the distal lower extremities in diabetic patients, often complicated by infection, and involving muscle and bone tissues in severe cases. Diabetic foot is one of the most serious chronic complications of diabetes with high morbidity and mortality. The treatment of chronic wounds of diabetic foot is difficult, long-term and expensive, which brings a heavy burden to patients, families and society.

At present, local wounds of diabetic foot are mostly treated by covering with gauze or some functional dressings after debridement. There are problems of low cure rate, long treatment time and high cost during the treatment. For chronic wounds of diabetic foot, healing is a slow process. Therefore, a long-acting biomaterial is required to work slowly to promote wound healing. Leucocyte and platelet rich fibrin (L-PRF) is a second-generation platelet gel product, which can promote the healing of diabetic foot ulcer, but the treatment effect needs to be improved. Therefore, there is an urgent need for a medicament with excellent treatment effect and high safety.

### SUMMARY

In order to solve the above problems, the present disclosure provides use of A-PRF in preparing a medicament for the treatment of diabetic foot ulcer. The medicament prepared from the A-PRF shortens the healing period of the diabetic foot ulcer, improves the healing rate, and has an excellent treatment effect and high safety.

To achieve the above objective, the present disclosure provides the following technical solutions:

The present disclosure provides use of A-PRF in preparing a medicament for the treatment of diabetic foot ulcer.

The present disclosure further provides a medicament for the treatment of diabetic foot ulcer, and the medicament includes A-PRF and pharmaceutically acceptable excipients.

The present disclosure has the following beneficial effects:

The present disclosure provides use of A-PRF in preparing a medicament for the treatment of diabetic foot ulcer. The A-PRF provided by the present disclosure has a loose reticular fibrin structure, can be rich in more platelets and leukocytes, can release a plurality of growth factors and cytokines more persistently, and is more beneficial to tissue regeneration and wound repair. The medicament prepared from the A-PRF shortens the healing period of the diabetic foot ulcer, improves the healing rate, and has an excellent treatment effect and high safety.

### BRIEF DESCRIPTION OF THE DRAWINGS

FIG. 1 is a picture of a wound of a patient in Case 1 of Application Example 1 taken on Nov. 30, 2020;

FIG. 2 is a picture of a wound of a patient in Case 1 of Application Example 1 taken on Dec. 15, 2020;

FIG. 3 is a picture of a wound of a patient in Case 1 of Application Example 1 taken on Feb. 7, 2021;

FIG. 4 is a picture of a wound of a patient in Case 2 of Application Example 1 taken on Sep. 1, 2020;

FIG. 5 is a picture of a wound of a patient in Case 2 of Application Example 1 taken on Sep. 14, 2020;

FIG. 6 is a picture of a wound of a patient in Case 2 of Application Example 1 taken on Oct. 13, 2020;

FIG. 7 is a picture of a wound of a patient in Case 3 of Application Example 1 taken on May 8, 2021;

FIG. 8 is a picture of a wound of a patient in Case 3 of Application Example 1 taken on May 25, 2021;

FIG. 9 is a picture of a wound of a patient in Case 3 of Application Example 1 taken on Jun. 3, 2021;

FIG. 10 is a picture of a wound of a patient in Case 4 of Application Example 1 taken on Sep. 13, 2021;

FIG. 11 is a picture of a wound of a patient in Case 4 of Application Example 1 taken on Sep. 16, 2021;

FIG. 12 is a picture of a wound of a patient in Case 4 of Application Example 1 taken on Sep. 26, 2021;

FIG. 13 is a picture of a wound of a patient in Case 5 of Comparative Example 1 taken on Aug. 21, 2020;

FIG. 14 is a picture of a wound of a patient in Case 5 of Comparative Example 1 taken on Sep. 2, 2020;

FIG. 15 is a picture of a wound of a patient in Case 5 of Comparative Example 1 taken on Nov. 21, 2020;

FIG. 16 is a picture of a wound of a patient in Case 6 of Comparative Example 1 taken on Sep. 28, 2020;

FIG. 17 is a picture of a wound of a patient in Case 6 of Comparative Example 1 taken on Oct. 15, 2020;

FIG. 18 is a picture of a wound of a patient in Case 6 of Comparative Example 1 taken on Dec. 23, 2020; and

FIG. 19 shows Kaplan-Meier curves of 60 patients in Application Example 1 and Comparative Example 1.

### DETAILED DESCRIPTION OF THE EMBODIMENTS

The present disclosure provides use of A-PRF in preparing a medicament for the treatment of diabetic foot ulcer.

The A-PRF provided by the present disclosure has a loose reticular fibrin structure, can be rich in more platelets and leukocytes, can release a plurality of growth factors and cytokines more persistently, and is more beneficial to tissue regeneration and wound repair. The medicament prepared from the A-PRF shortens the healing period of the diabetic foot ulcer, improves the healing rate, and has an excellent treatment effect and high safety.

The present disclosure further provides a medicament for the treatment of diabetic foot ulcer, and the medicament includes A-PRF and pharmaceutically acceptable excipients. In the present disclosure, the A-PRF may preferably include A-PRF prepared from autologous blood.

The present disclosure preferably further provides a preparation method of the foregoing A-PRF, including the following steps: centrifuging the blood at 1,500 rpm for 14 min, and separating an intermediate gel layer as the A-PRF. In the present disclosure, the blood may preferably include autologous blood; more preferably, the blood may include autologous blood collected within 1 min. The present disclosure uses the autologous blood to prepare the A-PRF, which can avoid immune rejection of foreign blood, with high safety.

To further describe the present disclosure, the use of A-PRF in preparing a medicament for the treatment of diabetic foot ulcer provided by the present disclosure will be described below in detail with reference to examples, but they may not be construed as limiting the protection scope of the present disclosure.

### Example 1

The A-PRF was prepared according to the following steps:

in strict accordance with aseptic operation, 5 mL of venous blood was placed in an anticoagulant-free vacuum glass tube, shaken well, and put in a balanced centrifuge immediately (within 1 min after blood collection); the tube was centrifuged at 1,500 rpm for 14 min at room temperature and left to stand, the blood samples were divided into three layers, the supernatant was discarded, the bottom red blood cell debris was removed, and the intermediate gel layer was collected to obtain the A-PRF.

The prepared A-PRF was left to stand in a dry and sterile bending tray, and placed between two sterile gauze layers to squeeze, namely, an A-PRF film was made, which was evenly spread or packed on the wound surface of the diabetic foot; the surface was covered with a sterile gauze impregnated with 1% ethacridine, and the outer layer was dressed with sterile gauzes.

### Application Example 1

A-PRF was prepared from part of the blood from 30 patients with diabetic foot ulcer according to the method of Example 1, and the prepared A-PRF films were applied to the wounds of the 30 patients with diabetic foot ulcer according to the method of Example 1. The wounds were checked once every 2-3 days, and the A-PRF films were changed every 10 days.

Typical cases were as follows:

Case 1:

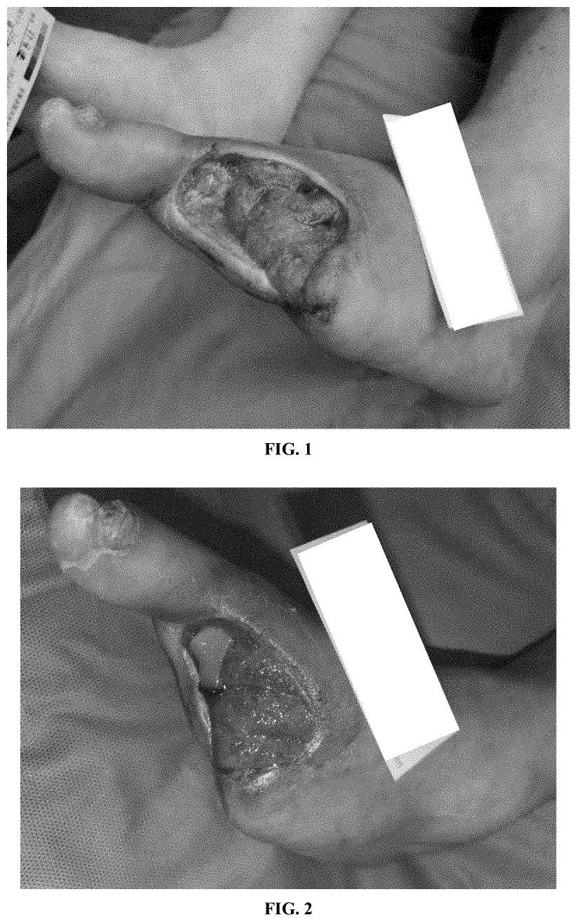

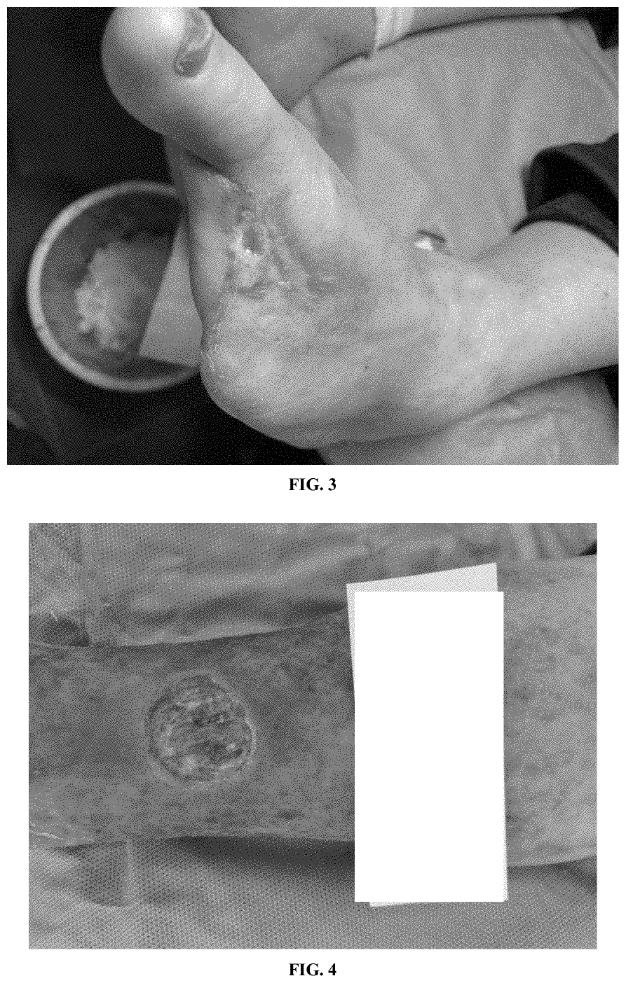

A 72-year-old female patient with chronic wound of diabetic foot (Wagner grade 3) was admitted to a hospital because of hyperglycemia for 20 years and bilateral foot ulceration for two years. The wound was treated with vacuum-assisted closure therapy and the above A-PRF successively, and photographed on Nov. 30, 2020 (FIG. 1), Dec. 15, 2020 (FIG. 2), and Feb. 7, 2021 (FIG. 3), respectively. The A-PRF treatment began on Dec. 15, 2020. The wound area was 17 cm². The wound was healed on Feb. 10, 2021, and the healing period was 57 days.

Case 2:

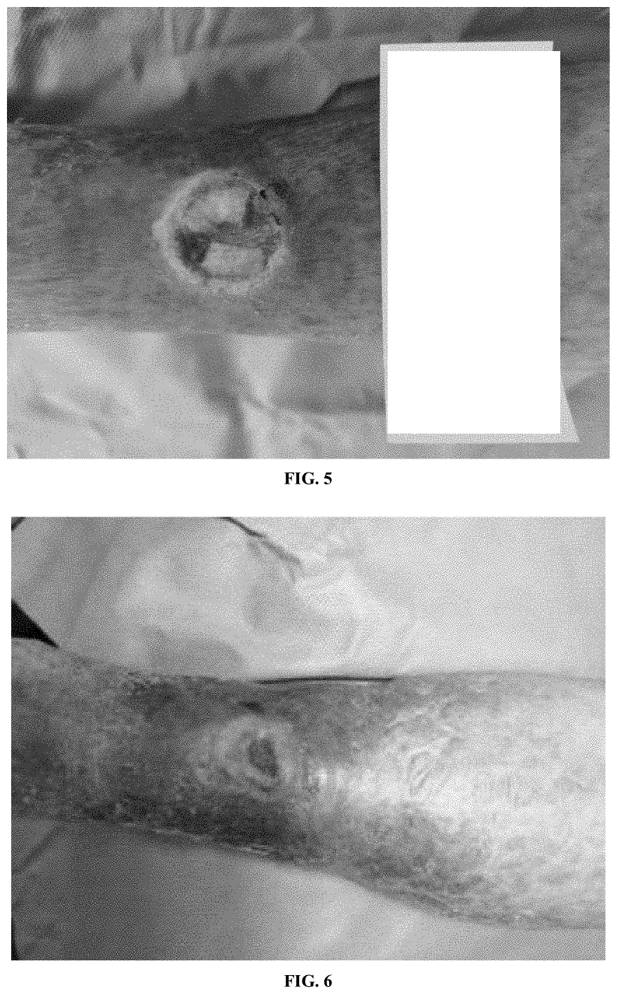

A 93-year-old female patient with chronic wound of diabetic foot (Wagner grade 2) was admitted to a hospital because of hyperglycemia for 18 years and left pretibial skin ulceration for a month. The wounds were treated with the above A-PRF, and the photos were taken on Sep. 1, 2020 (FIG. 4), Sep. 14, 2020 (FIG. 5), and Oct. 13, 2020 (FIG. 6), respectively. The A-PRF treatment began on Sep. 1, 2020. The wound area was 7 cm². The wound was healed on Oct. 20, 2020, and the healing period was 49 days.

Case 3:

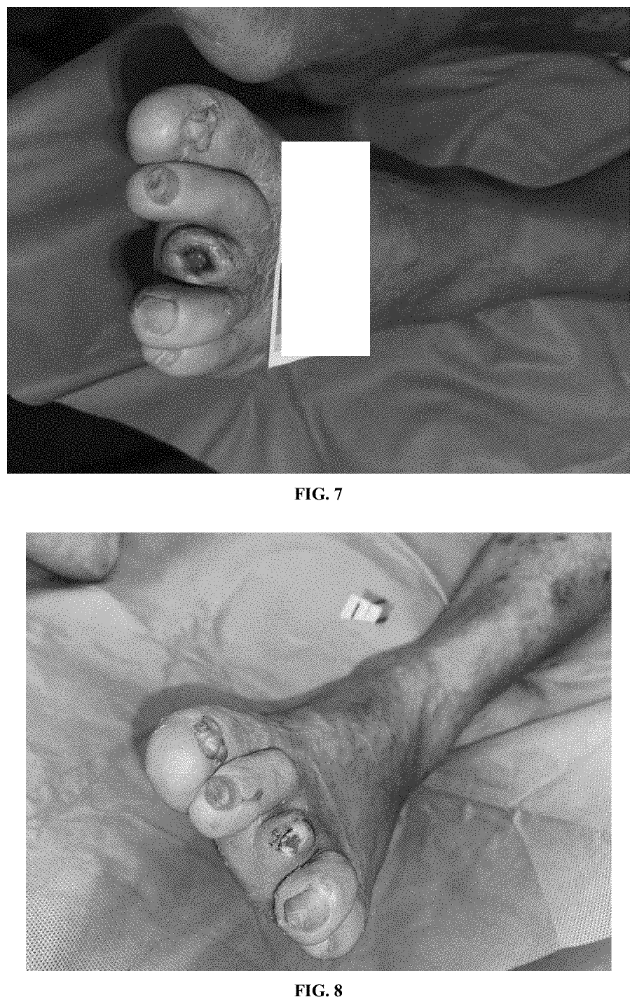

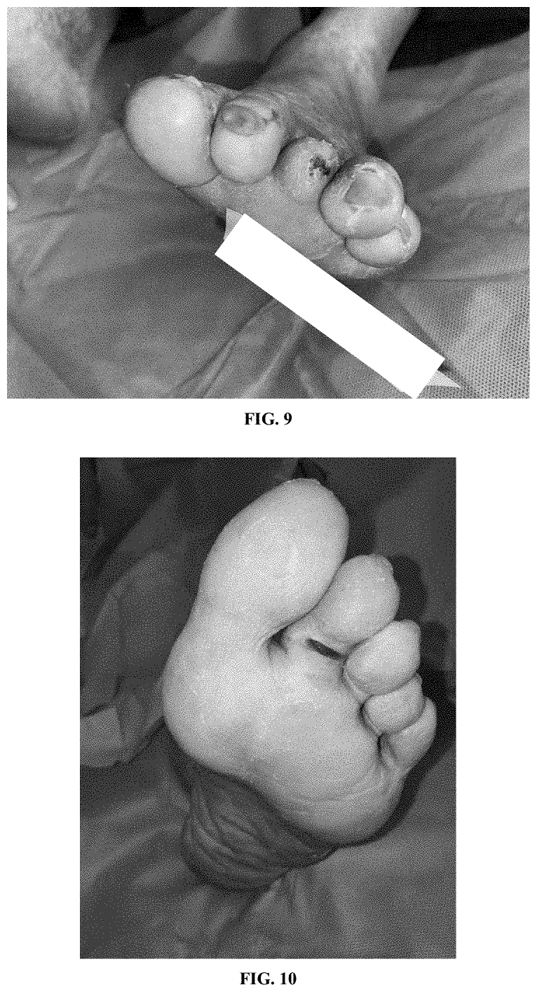

A 65-year-old medium-elderly male with chronic wound of diabetic foot (Wagner grade 3) was admitted to a hospital because of hyperglycemia for 30 years and bilateral foot ulceration for one year. The left foot wound was treated with A-PRF, and the photos were taken on May 8, 2021 (FIG. 7), May 25, 2021 (FIG. 8), and Jun. 3, 2021 (FIG. 9), respectively. The A-PRF treatment began on May 8, 2021. The wound area was 3 cm². The wound was healed on Jun. 3, 2021, and the healing period was 26 days.

Case 4:

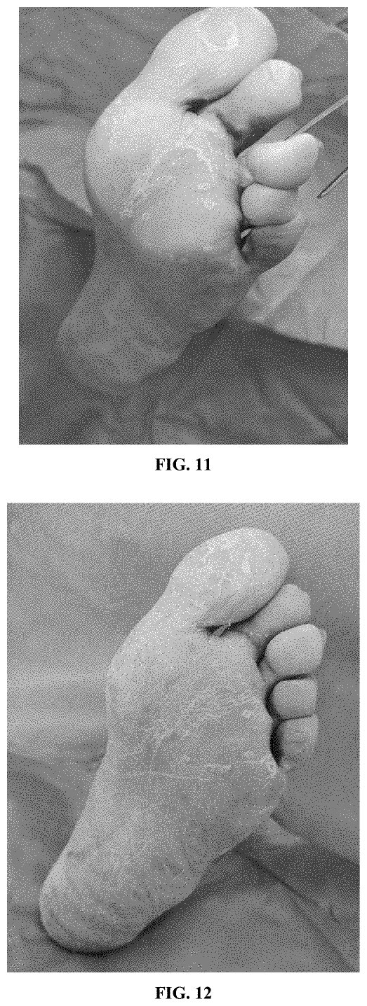

A 70-year-old senile male with chronic wound of diabetic foot (Wagner grade 2) was admitted to a hospital because of hyperglycemia for more than 10 years and left foot ulceration for one month. The left foot wound was treated with A-PRF, and the photos were taken on Sep. 13, 2021 (FIG. 10), Sep. 16, 2021 (FIG. 11), and Sep. 26, 2021 (FIG. 12), respectively. The A-PRF treatment began on Sep. 13, 2021. The wound area was 1 cm². The wound was healed on Sep. 26, 2021, and the healing period was 13 days.

### Comparative Example 1

L-PRF was prepared from part of the blood of 30 patients with diabetic foot ulcer and similar conditions as in Application Example 1, and the prepared L-PRF was left to stand in a dry and sterile bending tray, and placed between two sterile gauze layers to squeeze, namely, an A-PRF film was made, which was evenly spread or packed on the wound surface of the 30 patients with diabetic foot; the surface was covered with a sterile gauze impregnated with 1% ethacridine, and the outer layer was dressed with sterile gauzes. The wounds were checked once every 2-3 days, and the L-PRF films were changed every 7 days.

The L-PRF was prepared according to the following steps:

in strict accordance with aseptic operation, 5 mL of venous blood was placed in an anticoagulant-free vacuum glass tube, shaken well, and put in a balanced centrifuge immediately (within 1 min after blood collection); the tube was centrifuged at 3,000 rpm for 10 min at room temperature and left to stand, the blood samples were divided into three layers, the supernatant was discarded, the bottom red blood cell debris was removed, and the intermediate gel layer was collected to obtain the L-PRF.

Typical cases were as follows:

Case 5:

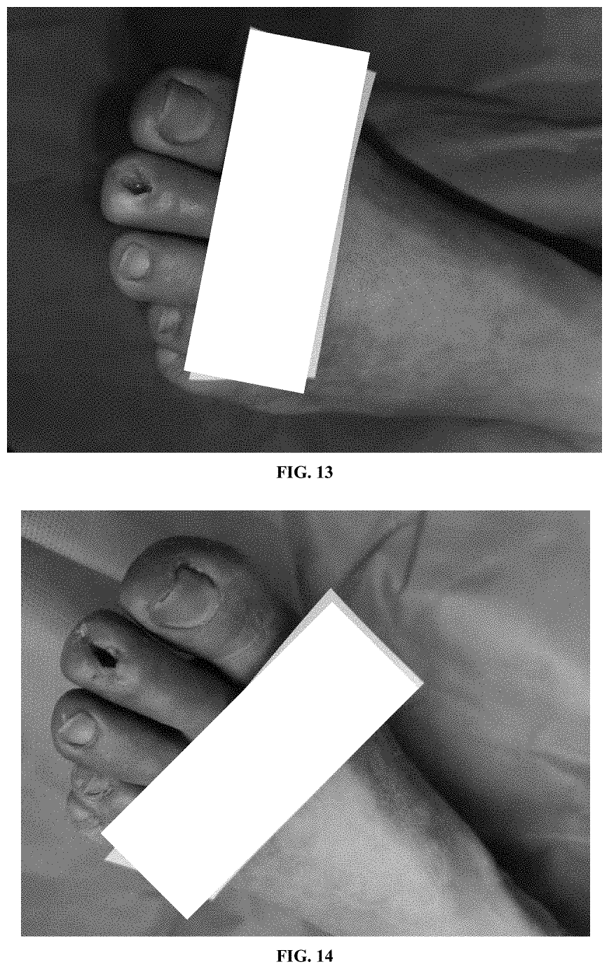

A 76-year-old senile female with chronic wound of diabetic foot (Wagner grade 3) was admitted to a hospital because of hyperglycemia for more than 20 years and left foot ulceration for two months. The left foot wound was treated with L-PRF, and the photos were taken on Aug. 21, 2020 (FIG. 13), Sep. 2, 2020 (FIG. 14), and Nov. 21, 2020 (FIG. 15), respectively. The L-PRF treatment began on Aug. 21, 2020. The wound area was 1 cm². No wound healing was observed after three months.

Case 6:

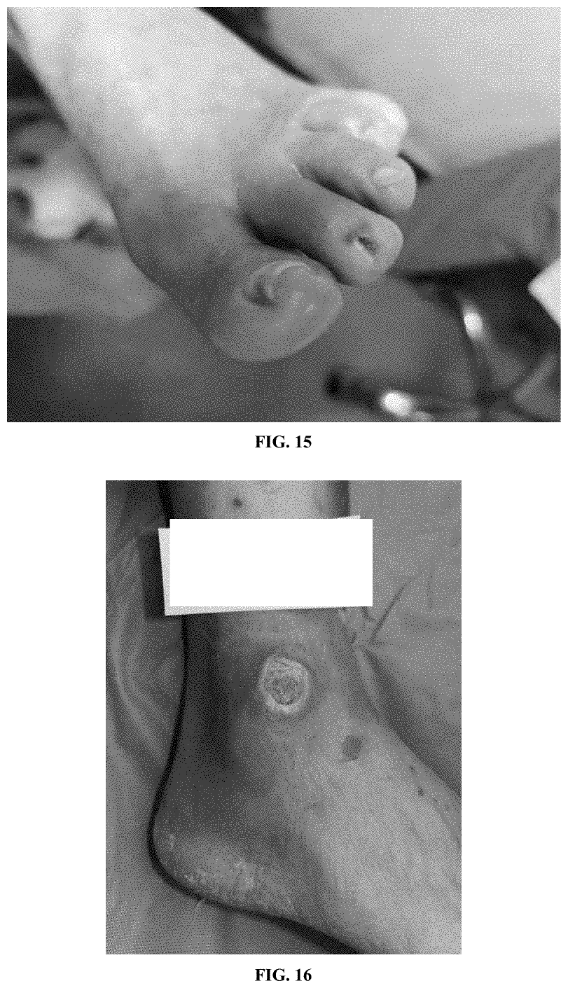

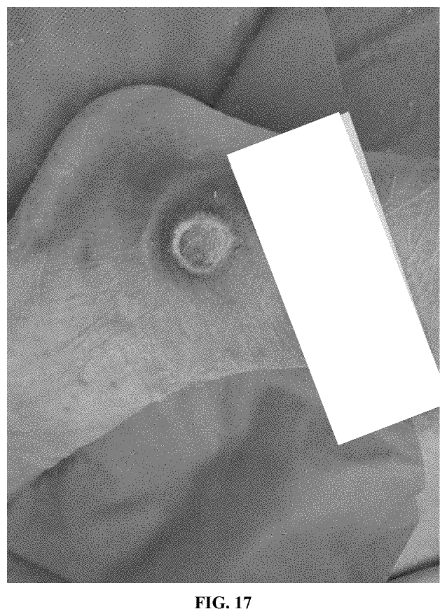

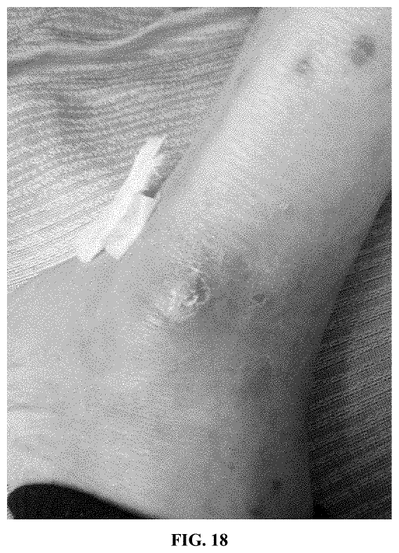

A 72-year-old senile male with chronic wound of diabetic foot (Wagner grade 2) was admitted to a hospital because of hyperglycemia for more than 13 years and left lateral malleolus ulceration for two months. The wound was treated with L-PRF, and the photos were taken on Sep. 28, 2020 (FIG. 16), Oct. 15, 2020 (FIG. 17), and Dec. 23, 2020 (FIG. 18), respectively. The L-PRF treatment began on Sep. 28, 2020. The wound area was 3 cm². The wound was healed on Dec. 23, 2020, and the healing period was 86 days.

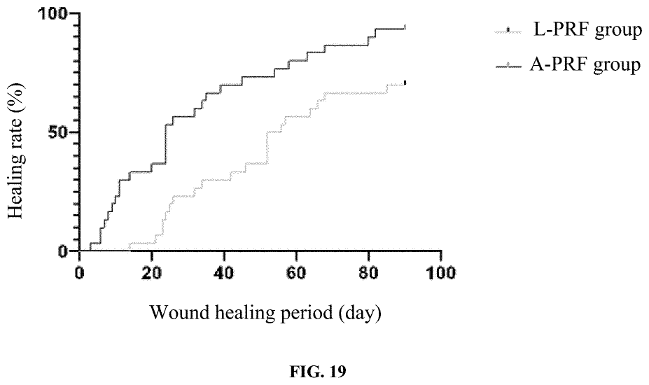

The treatment effects of a total of 60 patients in Application Example 1 and Comparative Example 1 were observed and summarized. The results are shown in Table 1 and FIG. 19.

TABLE 1
Treatment effects of different patients

| | Wound healing | Median healing period (upper |
|---|---|---|
| Group | rate (%) | and lower quartiles) (day) |
| Application Example 1 | 93.4% | 24 (11-52) |
| Comparative Example 1 | 70% | 52 (28-74) |

NOTE:
The wound healing rate in Table 1 = the number of healed patients/30 × 100%; since the healing period does not conform to the normal distribution, it is expressed by the median healing period (upper and lower quartiles), and the median healing period = the median of healing period.

As seen from Table 1 and FIG. 19, there were statistically significant differences in wound healing rate and median healing period between the dressing change method provided by the present disclosure and that in Comparative Example 1, the wound healing rate of diabetic foot ulcer for three months was increased by 33.4%, and the median healing period was shortened by 53.8%; in addition, the dressing change method provided by the present disclosure significantly reduced the dressing change frequency compared with Comparative Example 1, which could further alleviate the pain of the patients during blood collection and save treatment costs. In addition, Kaplan-Meier curves showed the accumulation of the healing rate of both groups over time, and the log-rank test indicated that the healing of the A-PRF group was significantly better than that of the L-PRF group (X²=9.339, P=0.0022).

In conclusion, the medicament prepared from the A-PRF provided by the present disclosure shortens the healing period of the diabetic foot ulcer, improves the healing rate, and has an excellent treatment effect and high safety.

Although the present disclosure has been set forth as above in preferred examples, they are not intended to limit the present disclosure. Those skilled in the art can make various alterations and modifications without departing from the spirit and scope of the present disclosure. Therefore, the protection scope of the present disclosure should be subject to the protection scope defined by the claims.

What is claimed is:

1. A method for treating diabetic foot ulcer, the method comprising using advanced platelet rich fibrin (A-PRF), wherein the A-PRF is prepared according to the following steps: centrifuging blood at 1,500 rpm for 14 minutes, and separating an intermediate gel layer as the A-PRF, wherein the blood is autologous blood collected within 1 minute.

2. A medicament for the treatment of diabetic foot ulcer, wherein the medicament comprises A-PRF and pharmaceutically acceptable excipients.
